(19) United States
(12) Patent Application Publication
(10) Pub. No.: US 2022/0319666 A1
(43) Pub. Date: Oct. 6, 2022

(54) ELECTRONIC DEVICE FOR PROVIDING PLAN, AND METHOD OF OPERATING THE SAME

(71) Applicant: Samsung Electronics Co., Ltd., Suwon-si, Gyeonggi-do (KR)

(72) Inventors: Taehun KIM, Suwon-si (KR); Jidon YEO, Suwon-si (KR); Haanju YOO, Suwon-si (KR); Byeongchan LEE, Suwon-si (KR); Sangwoong LEE, Suwon-si (KR)

(21) Appl. No.: 17/599,887

(22) PCT Filed: Apr. 13, 2021
(86) PCT No.: PCT/KR2021/004632

§ 371 Date: Sep. 29, 2021

(30) Foreign Application Priority Data
Apr. 16, 2020 (KR) 10-2020-0046266
Nov. 12, 2020 (KR) 10-2020-0151114
Jan. 7, 2021 (KR) 10-2021-0002207

(51) Int. Cl. G16H 20/60

(52) U.S. Cl. CPC G16H 20/60

(57) ABSTRACT

A method, performed by an electronic device, of providing a plan, is provided. The method includes obtaining a plan including a plurality of items, predicting, for one or more points of time, a storage amount of a first ingredient to be consumed over time based on the plan, measuring the storage amount of the first ingredient at the one or more points of time, detecting a difference between the measured and predicted values at a first point of time from among the one or more points of time, and providing the plan by modifying the plan to reduce the difference after the first point of time, when the detected difference is greater than or equal to a reference value.

### CROSS-REFERENCE TO RELATED APPLICATION(S)

This application is a U.S. National Stage application under 35 U.S.C. § 371 of an International application number PCT/KR2021/004632, filed on Apr. 13, 2021, which is based on and claims priority of a Korean patent application number 10-2020-0046266, filed on Apr. 16, 2020, in the Korean Intellectual Property Office, of a Korean patent application number 10-2020-0151114, filed on Nov. 12, 2020, in the Korean Intellectual Property Office, and of a Korean patent application number 10-2021-0002207, filed on Jan. 7, 2021, in the Korean Intellectual Property Office, the disclosure of each of which is incorporated by reference herein in its entirety.

### BACKGROUND

### 1. Field

The disclosure relates to an electronic device for creating and providing a plan to a user, and a method of operating the same.

### 2. Description of Related Art

A user may make and carry out various plans to stay healthy and achieve personal goals. An electronic device according to an embodiment may create and provide various types of plans, e.g., a diet plan and an exercise plan, to a user based on a preference or a goal of the user.

The plan provided to the user may be freely changed based on a user input. However, the user may experience inconvenience to change the plan appropriately for the preset goal considering features (e.g., nutrients and calories) of all items (e.g., recipes) included in the plan. Therefore, a method capable of providing a plan suitable for a user in various circumstances is provided below.

The above information is presented as background information only to assist with an understanding of the disclosure. No determination has been made, and no assertion is made, as to whether any of the above might be applicable as prior art with regard to the disclosure.

### SUMMARY

Aspects of the disclosure are to address at least the above-mentioned problems and/or disadvantages and to provide at least the advantages described below. Accordingly, an aspect of the disclosure is to provide an electronic device for providing a plan, and a method of operating the same.

Another aspect of the disclosure is to provide a computer-readable recording medium having recorded thereon a computer program for executing the method. Technical problems to be solved are not limited to the above-described problems, and other technical problems may exist.

Additional aspects will be set forth in part in the description which follows and, in part, will be apparent from the description, or may be learned by practice of the presented embodiments.

In accordance with an aspect of the disclosure, a method, performed by an electronic device, of providing a plan is provided. The method includes obtaining a plan including a plurality of items, predicting, for one or more points of time, a storage amount of a first ingredient to be consumed over time based on the plan, measuring the storage amount of the first ingredient at the one or more points of time, detecting a difference between the measured and predicted values at a first point of time from among the one or more points of time, and providing the plan by modifying the plan to reduce the difference after the first point of time, when the detected difference is greater than or equal to a reference value.

In accordance with another aspect of the disclosure, a method, performed by an electronic device, of providing a plan is provided. The method includes creating a plan including a plurality of items, based on a preset condition, selecting one of the plurality of items based on an external input signal, obtaining a first candidate item for replacing the selected item, identifying an item to be replaced to satisfy the preset condition from among the plurality of items, when the preset condition is not satisfied after the selected item is replaced by the first candidate item, obtaining a second candidate item for replacing the identified item, and modifying the plan based on the first and second candidate items, and displaying the modified plan.

In accordance with another aspect of the disclosure, an electronic device for providing a plan is provided. The electronic device includes an inputter for receiving an external input signal, a memory for storing one or more instructions, and at least one processor for executing the one or more instructions stored in the memory, wherein the at least one processor obtains a plan including a plurality of items, predicts, for one or more points of time, a storage amount of a first ingredient to be consumed over time based on the plan, measures the storage amount of the first ingredient at the one or more points of time, detects a difference between the measured and predicted values at a first point of time from among the one or more points of time, and provides the plan by modifying the plan to reduce the difference after the first point of time, when the detected difference is greater than or equal to a reference value.

In accordance with another aspect of the disclosure, an electronic device for providing a plan is provided. The electronic device includes an inputter for receiving an external input signal, a memory for storing one or more instructions, and at least one processor for executing the one or more instructions stored in the memory, wherein the at least one processor creates a plan including a plurality of items, based on a preset condition, selects one of the plurality of items based on the external input signal, obtains a first candidate item for replacing the selected item, identifies an item to be replaced to satisfy the preset condition from among the plurality of items, when the preset condition is not satisfied after the selected item is replaced by the first candidate item, obtains a second candidate item for replacing the identified item, and modifies the plan based on the first and second candidate items, and provides the modified plan.

In accordance with another aspect of the disclosure, a recording medium having recorded thereon a computer program for executing the method of the first aspect is provided.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a diagram showing an example of a system for providing a plan, according to an embodiment of the disclosure;

FIG. 2 is a diagram showing an example of inputting information to create a plan, according to an embodiment of the disclosure;

FIG. 3 is a block diagram for describing an example of creating a plan, according to an embodiment of the disclosure;

FIG. 4 is a diagram showing an example of replacing an item included in a plan, based on an input of a user, according to an embodiment of the disclosure;

FIG. 5 is a block diagram showing an example of modifying a plan, according to an embodiment of the disclosure;

FIG. 6 is a graph showing an example of predicting a storage amount of an ingredient based on a plan, according to an embodiment of the disclosure;

FIG. 7 is a graph showing an example of a measured value of a storage amount of an ingredient based on a plan, according to an embodiment of the disclosure;

FIG. 8 is a graph showing an example of a measured value of a storage amount of an ingredient based on a plan, according to an embodiment of the disclosure;

FIG. 9 is a block diagram of an electronic device according to an embodiment of the disclosure;

FIG. 10 is a detailed block diagram of an electronic device according to an embodiment of the disclosure;

FIG. 11 is a flowchart of a method of providing a plan, according to an embodiment of the disclosure; and

FIG. 12 is a flowchart of a method of providing a plan, according to an embodiment of the disclosure.

Throughout the drawings, like reference numerals will be understood to refer to like parts, components, and structures.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise. Thus, for example, reference to “a component surface” includes reference to one or more of such surfaces.

Throughout the specification, when an element is referred to as being “connected to” another element, the element can be “directly connected to” the other element or be “electrically connected to” the other element via an intervening element. The terms “comprises”, “comprising”, “includes” and/or “including”, when used herein, specify the presence of stated elements, but do not preclude the presence or addition of one or more other elements, unless otherwise indicated herein.

Artificial intelligence (AI)-related functions according to the disclosure are performed using a processor and a memory. The processor may include one or more processors. In this case, each of the one or more processors may be a general-purpose processor such as a central processing unit (CPU), an application processor (AP), or a digital signal processor (DSP), a dedicated graphics processor such as a graphics processing unit (GPU) or a vision processing unit (VPU), or a dedicated AI processor such as a neural processing unit (NPU). The one or more processors control input data to be processed according to a predefined operation rule or AI model stored in the memory. Alternatively, when the one or more processors are dedicated AI processors, the dedicated AI processors may be designed in a hardware structure specialized for processing a specific AI model.

The predefined operation rule or AI model is characterized by being made through training. Herein, being made through training means that a basic AI model is trained based on a learning algorithm by using a plurality of pieces of training data and thus a predefined operation rule or AI model configured to achieve a desired feature (or purpose) is made. The training may be performed directly by a device having an AI function according to the disclosure, or via a separate server and/or system. Examples of the learning algorithm may include supervised learning, unsupervised learning, semi-supervised learning, and reinforcement learning, but are not limited thereto.

The AI model may include a plurality of neural network layers. Each of the plurality of neural network layers has a plurality of weight values and performs neural network computation through computation between a computation result of a previous layer and the plurality of weight values. The plurality of weight values of the plurality of neural network layers may be optimized based on a result of training the AI model. For example, the plurality of weight values may be updated to reduce or minimize a loss or cost value obtained by the AI model during the training process. An artificial neural network may include, for example, a convolutional neural network (CNN), a deep neural network (DNN), a recurrent neural network (RNN), a restricted Boltzmann machine (RBM), a deep belief network (DBN), a bidirectional recurrent deep neural network (BRDNN), or a deep Q-network, but is not limited thereto.

Reference will now be made in detail to embodiments of the disclosure, examples of which are illustrated in the accompanying drawings.

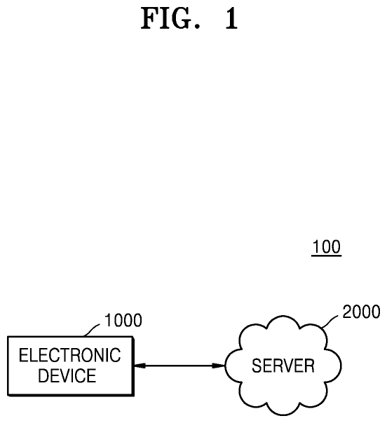

FIG. 1 is a diagram showing an example of a system for providing a plan, according to an embodiment of the disclosure.

Referring to FIG. 1, a system 100 for providing a plan, according to an embodiment, may include an electronic device 1000 and a server 2000.

According to a request of a user, the electronic device 1000 according to an embodiment may receive, from the server 2000, data required to create a plan, create the plan related to various activities of the user, and provide the plan to the user.

The plan according to an embodiment may be created by placing one or more items in a timetable for a healthy life and a goal of the user. The plan according to an embodiment may include a plan related to various activities of the user, e.g., a diet plan, an exercise plan, a study plan, or a work plan, depending on a purpose thereof.

For example, when the plan is a diet plan, the plan may be created by placing one or more recipes as the items in the timetable. Alternatively, when the plan is an exercise plan, the plan may be created by placing one or more exercises (e.g., running, cycling, and Pilates) as the items in the timetable. Without being limited to the above-described example, the plan may be related to various activities of the user.

The electronic device 1000 according to an embodiment may be implemented in a variety of forms. For example, the electronic device 1000 described herein may include a digital camera, a smartphone, a laptop computer, a tablet personal computer (PC), an e-book reader, a digital broadcast receiver, a personal digital assistant (PDA), a portable multimedia player (PMP), a navigation system, a moving picture experts group audio layer 3 (MP3) player, a vehicle, a robotic vacuum cleaner, a refrigerator, or a television (TV), but is not limited thereto. The electronic device 1000 described herein may be a device wearable by the user. The wearable device may include at least one of an accessory-type device (e.g., a watch, a ring, a wrist band, an ankle band, a necklace, glasses, or contact lenses), a head-mounted device (HMD), a fabric- or clothes-integrated device (e.g., electronic clothing), a body-attachable device (e.g., a skin pad)), or an implantable device (e.g., an implantable circuit), but is not limited thereto.

According to a request of the electronic device 1000, the server 2000 according to an embodiment may transmit, to the electronic device 1000, the data required to create the plan. For example, the server 2000 may receive, from the electronic device 1000, various types of user information, e.g., a preference, a goal, and a health status of the user, and thus transmit, to the electronic device 1000, item information indicating one or more items for creating a plan suitable for the user. The electronic device 1000 according to an embodiment may create a plan by placing at least one of the items received from the server 2000, in the plan.

Without being limited to the above-described example, the server 2000 may transmit, to the electronic device 1000, various types of data required by the electronic device 1000 to create the plan.

The item information according to an embodiment may include information about features of the items. For example, when the items are recipes, the item information may include various types of information usable to place the items in the plan suitably for the user, e.g., nutrients, glycemic indices, cooking methods, ingredient information, names, and purchase prices of the recipes.

The server 2000 according to an embodiment may execute some operations on behalf of the electronic device 1000. For example, when the request of the user is received from the electronic device 1000, the server 2000 may create the plan on behalf of the electronic device 1000 and transmit the plan to the electronic device 1000. The electronic device 1000 according to an embodiment may provide, to the user, the plan received from the server 2000.

According to an embodiment, the user may perform various activities such as exercise, diet, or work with reference to the plan, and thus various ingredients (e.g., foods or materials) required for the activities may be consumed. For example, the user may make meals based on recipes included in the plan, and thus foods included in the recipes may be consumed.

According to an embodiment, when the ingredients have use-by dates, it is desirable to consume the ingredients within the use-by dates, and thus the plan may be created considering the use-by dates of the ingredients. The plan according to an embodiment is not limited to the user information and may be created by further using the use-by dates of the ingredients usable for the plan.

According to an embodiment, the plan may be created by determining consumption rates of the ingredients based on the use-by dates of the ingredients. For example, when the plan is a diet plan, the plan may be created by placing one or more recipes in a timetable to consume one or more foods within use-by dates thereof.

The ingredients according to an embodiment may be classified based on types of the ingredients, and use-by dates thereof may be independently determined to create or modify the plan. The ingredients according to an embodiment may be classified according to various criteria, e.g., vegetables, meat, and dairy products, and a use-by date per category may be determined to create or modify the plan. In this case, although the ingredients may be individually used for each item of the plan, the use-by date usable to modify the plan according to an embodiment may be determined per upper category including a plurality of ingredients (e.g., vegetables, meat, or dairy products). Without being limited to the above-described example, the plan may be created or modified by setting the use-by dates for various ingredients classified according to various criteria.

The use-by date according to an embodiment refers to a date by which each ingredient is consumed in good quality and, for example, the use-by date of a food may be determined based on an expiration date of the food. Unlike the expiration date, the use-by date according to an embodiment may be changed in real time depending on a storage state of the food. For example, when raw meat is stored at room temperature for a long period of time, the use-by date may be changed to a point of time earlier than the expiration date. Without being limited to the above-described example, the use-by dates of the ingredients may be determined in various manners.

When the use-by date of each ingredient is changed by more than a reference value, the plan according to an embodiment may be modified based on the changed use-by date. According to an embodiment, the use-by date of the ingredient may be changed in real time depending on a storage state of the ingredient, and the plan may be modified based on the changed use-by date.

The electronic device 1000 according to an embodiment may detect one or more ingredients stored by the user and determine a storage amount and a use-by date of each ingredient. For example, the storage amount and the use-by date of each food may be determined by detecting weights, appearances, barcodes, or quick response (QR) codes of one or more foods stored in a refrigerator, or based on food information detected using a purchase list. Alternatively, the user may input, directly to the electronic device 1000, information about the ingredients stored by the user. Without being limited to the above-described example, the storage amounts and the use-by dates of one or more ingredients may be determined in various manners and the plan may be created or modified based on the storage amounts and the use-by dates.

According to an embodiment, consumption amounts of the ingredients predicted based on the plan may differ from amounts of the ingredients consumed due to activities of the user.

For example, when the plan is a diet plan, the user may make meals by using more foods than amounts preset by the plan. In this case, the electronic device 1000 according to an embodiment may measure the storage amounts of the ingredients in real time, modify the plan suitably for the measured values, and re-provide the plan to the user. The electronic device 1000 according to an embodiment may modify a part of the plan set after a current point of time based on the currently measured storage amounts of the ingredients.

The electronic device 1000 according to an embodiment may compare the storage amount of each ingredient previously predicted based on the plan, to the measured storage amount of the ingredient, and modify the plan when a difference therebetween is greater than or equal to a reference value. For example, when the storage amount of the ingredient detected by the electronic device 1000 is greater than the previously predicted storage amount, the ingredient may not be easily completely consumed within the use-by date. Therefore, the electronic device 1000 may modify the plan to increase an amount of the ingredient to be consumed for the same period of time. Otherwise, when the storage amount of the ingredient detected by the electronic device 1000 is less than the previously predicted storage amount, the ingredient may run out at a point of time earlier than the use-by date. Therefore, the electronic device 1000 may modify the plan to reduce the amount of the ingredient to be consumed for the same period of time.

The electronic device 1000 according to an embodiment may detect the storage amounts of one or more ingredients to be consumed based on the plan, and repeatedly modify the plan based on the use-by dates of the ingredients to consume the ingredients at appropriate rates.

A range in which the plan according to an embodiment is modified may be determined based on an input or a preference of the user. According to an embodiment, a part of the plan, which is set after the current point of time and belongs to the set range, may be modified based on changes in the storage amounts of the ingredients. A degree to which the plan is modified may vary depending on the input and the preference of the user.

For example, the plan may be set to be modified from a point of time three days later than the current point of time, based on the input and the preference of the user. Alternatively, based on the input of the user, the plan may be preset to modify only a preset number of items. Without being limited to the above-described example, the range in which the plan is modified may be preset in various manners.

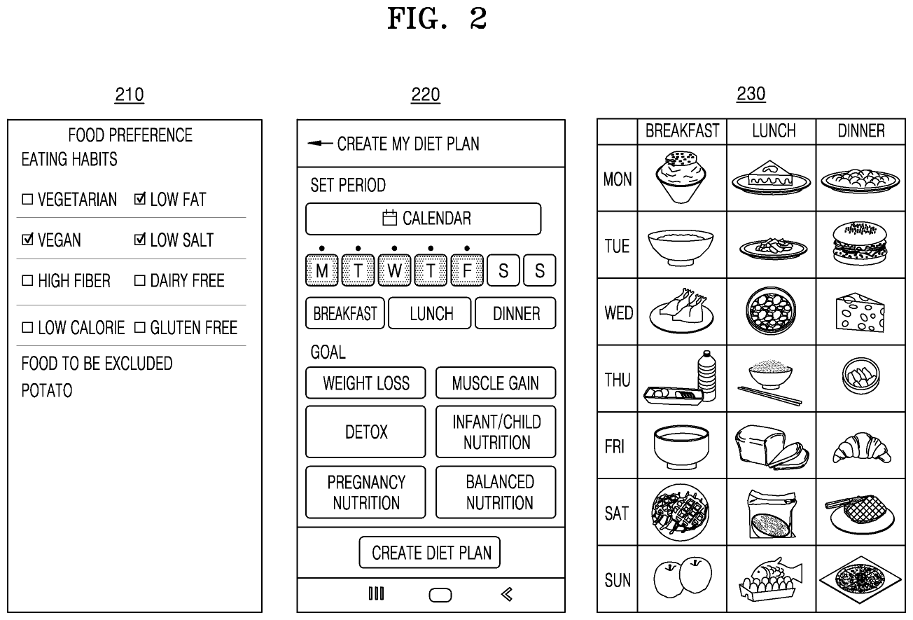

FIG. 2 is a diagram showing an example of inputting information to create a plan, according to an embodiment of the disclosure.

Referring to FIG. 2, an electronic device 1000 according to an embodiment may create and provide a plan 230 to a user based on a user input received through interfaces 210 and 220.

Through the interfaces 210 and 220, the electronic device 1000 according to an embodiment may receive, from the user, information required to create the plan. For example, the electronic device 1000 may receive, from the user, information about eating habits, foods to be excluded, a goal, etc. as information about features or conditions of items to be included in the plan.

The electronic device 1000 according to an embodiment may receive the information required to create the plan directly from the user, but is not limited thereto and may collect and use various types of user information to create the plan. For example, the electronic device 1000 may collect various types of user information, e.g., information about a plan previously created by the user, information about a health status of the user, information about a lifestyle of the user, information about foods currently usable by the user, and create the plan by using the collected user information.

The plan according to an embodiment may be created by placing one or more items to consume one or more ingredients stored by the user, within use-by dates.

Without being limited to the above-described example, the electronic device 1000 may select items to be placed in the plan, and obtain various types of user information usable to place the items, in various manners.

The electronic device 1000 according to an embodiment may create the plan based on information received from the user or various types of information collected about the user through the interfaces 210 and 220. The electronic device 1000 according to an embodiment may create the plan by transmitting the user information to the server 2000 and receiving, from the server 2000, one or more items to be included in the plan. For example, when the plan is a diet plan, the electronic device 1000 may create the plan by receiving information about various recipes from the server 2000 and placing the recipes in a 7-day breakfast, lunch, and dinner plan.

The electronic device 1000 according to an embodiment may select and place items to create the plan suitably for a condition and a goal set by the user through the interfaces 210 and 220. For example, when the user sets to exclude “potato” through the interface 210, the electronic device 1000 may create the plan 230 by selecting a plurality of recipes not using “potato”. The electronic device 1000 may create the plan 230 by selecting recipes not including “potato”, based on information about ingredients of each recipe.

Based on the information about the eating habits selected by the user through the interface 210, the electronic device 1000 may create the plan 230 capable of satisfying low-fat, vegan, and low-salt conditions. For example, the electronic device 1000 may create the plan 230 by placing the recipes to satisfy the condition, based on information about nutrients and ingredients of each recipe.

Furthermore, according to the goal selected by the user through the user interface 220, the electronic device 1000 may place the recipes based on information about nutrients and calories of each recipe. For example, when weight loss is set as the goal, the recipes may be placed in the plan 230 in such a manner that total calories of the recipes per day belong to a calorie range preset for “weight loss”, based on the information about the nutrients and calories of each recipe.

Without being limited to the above-described example, the electronic device 1000 may create the plan 230 based on various types of information set by the user.

The plan 230 according to an embodiment may be created suitably for the user based on a set of one or more scores of the plan 230. The scores according to an embodiment may be values representing suitability of the plan 230 for the user or a preference of the user for the plan 230. For example, the plan 230 may be created by placing one or more items in the plan 230 in such a manner that a final score corresponding to the set of one or more scores has the highest value. The final score according to an embodiment may be determined as a sum of the one or more scores, but is not limited thereto and may be determined in various manners.

The set of scores according to an embodiment may include scores calculated according to various criteria, e.g., a score representing a degree of overlap of items in the plan 230, a score representing a degree of satisfaction of nutritional requirements, a score representing a degree of diversity of cooking methods between items, and a score representing whether one or more ingredients are consumable within use-by dates. Each score according to an embodiment may be determined based on a pre-trained score model. The score model according to an embodiment may include a pre-trained artificial intelligence (AI) model or a rule-based model to obtain a score capable of representing suitability of the plan 230 for the user or a preference of the user for the plan 230. Without being limited to the above-described example, the score of the plan 230 according to an embodiment may be determined in various manners.

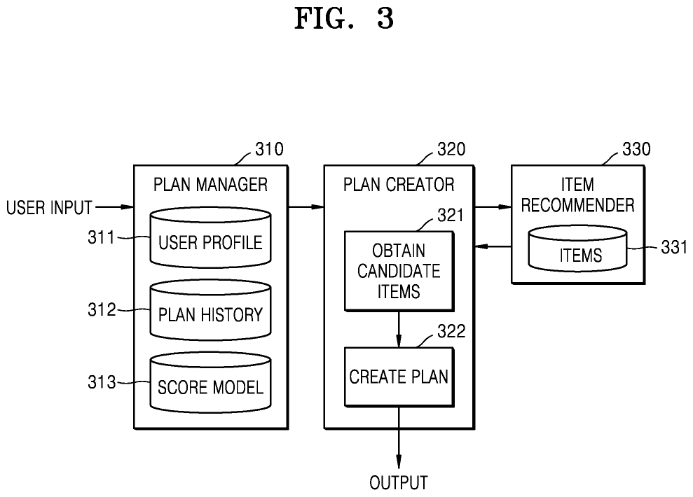

FIG. 3 is a block diagram for describing an example of creating a plan, according to an embodiment of the disclosure.

Referring to FIG. 3, an electronic device 1000 according to an embodiment may create a plan by using a plan manager 310, a plan creator 320, and an item recommender 330. At least one of the plan manager 310, the plan creator 320, and the item recommender 330 according to an embodiment may be executed by the electronic device 1000, and the others may be executed by the server 2000 or an external device.

The plan manager 310 according to an embodiment may collect and manage various types of user information used to create the plan. For example, the plan manager 310 may store and update a user profile 311, a plan history 312, a score model 313, and ingredient information 314 as the user information.

The user profile 311 according to an embodiment may include various types of user information. For example, the user profile 311 may include various types of user information, e.g., the age, height, gender, weight, health status, lifestyle, allergy information, ambient environment information, goal, and preference information of a user. The user profile 311 according to an embodiment may repeatedly updated based on the collected user information.

The plan history 312 according to an embodiment may include information about a plan previously provided to the user. According to an embodiment, one or more plans may be suggested to the user by the electronic device 1000, and a plan finally selected by the user from among the suggested plans may be used by the user. Therefore, the plan finally selected by the user may be determined as a plan most suitable for and most preferred by the user. The plan history 312 according to an embodiment may include information about the plan finally selected by the user, and be used when the electronic device 1000 provides a plan suitable for the user.

The score model 313 according to an embodiment may include one or more score models for determining one or more scores of the plan. The scores according to an embodiment may represent a preference of the user for the plan or suitability of the plan for the user, and the score model may be a model pre-trained to determine the scores.

For example, the score model 313 may include a model for determining various types of scores, e.g., a score representing a degree of overlap of items in the plan, a score representing a degree of satisfaction of nutritional requirements, a score representing a degree of diversity of cooking methods between items, and a score representing whether one or more ingredients stored by the user are consumable within use-by dates.

The score model 313 according to an embodiment may be repeatedly updated based on selection of the user in the plan. For example, when an item included in the plan is replaced by another item based on an input of the user, the other item selected by the user may be determined as being more preferred by the user than the item previously included in the plan. Therefore, the score model 313 may be updated to obtain a higher score of a plan including the selected other item compared to the previously included item. The score model 313 according to an embodiment may be repeatedly updated to reflect a user preference to create the plan based on the user profile 311 and the plan history 312. Without being limited to the above-described example, the score model 313 may be repeatedly updated based on various requests and inputs of the user for the plan.

The score model 313 according to an embodiment may be an AI model or a rule-based model, but is not limited thereto and may include models configured in various manners.

The ingredient information 314 according to an embodiment may include information about one or more ingredients to be consumed by the user to perform various activities based on the plan. For example, the ingredient information 314 may include information about storage amounts and use-by dates of one or more ingredients currently stored by the user.

The ingredient information 314 according to an embodiment may further include information about an ingredient usage history of the user. For example, when the ingredient usage history indicates that the user tends to use more beef than an amount set by a recipe, the plan may be created based on the tendency of the user to increase the amount of beef included in the recipe. Without being limited to the above-described example, the ingredient information 314 may include various types of information about the ingredients usable to create the plan.

The item recommender 330 according to an embodiment may collect one or more items 331 capable of configuring the plan, and transmit at least one of the collected items 331 to the plan creator 320 according to a request of the plan creator 320. According to a request of the plan creator 320, the item recommender 330 according to an embodiment may identify one or more items suitable to create the plan of the user from among the collected items 331, and transmit the identified one or more items to the plan creator 320.

The item recommender 330 according to an embodiment may repeatedly collect information about new items from outside. For example, when new recipes appear, e.g., when diet trends change or new cooking methods appear, the item recommender 330 repeatedly collect the new recipes through webpage search or by using an external data base storing recipes. Without being limited to the above-described example, the item recommender 330 may repeatedly collect and store various items in various manners.

The item recommender 330 according to an embodiment may identify items usable to create the plan from among the items 331 repeatedly collected from outside through search or the like, based on the user information, e.g., the user profile 311. For example, the item recommender 330 may identify recipes using foods edible by the user as the items usable to create the plan by excluding recipes using foods inedible by the user, based on the allergy information or the health status of the user which is included in the user profile 311. Alternatively, the item recommender 330 may identify recipes suitable for the user as the items usable to create the plan, based on a condition included in the user profile 311 and preset by the user to create the plan. In this case, the user profile 311 is information about the condition input by the user and preset to create the plan, and may further include, for example, the information obtained through the interfaces 210 and 220 of FIG. 2.

Without being limited to the above-described example, the item recommender 330 may identify the items usable to create the plan, in various manners.

The plan creator 320 according to an embodiment may create the plan according to a request of the user. The plan creator 320 according to an embodiment may obtain one or more candidate items from the item recommender 330 (see reference numeral 321), and create the plan based on the candidate items (see reference numeral 322).

The plan creator 320 according to an embodiment may obtain the one or more candidate items from the item recommender 330 (see reference numeral 321) by transmitting the user profile 311 received from the plan manager, to the item recommender 330 according to a plan creation request of the user.

The plan creator 320 according to an embodiment may create the plan (see reference numeral 322) by placing the one or more candidate items in the plan suitably for a user preference, based on the plan history 312 and the score according to the score model 313, which are received from the plan manager 310.

The plan creator 320 according to an embodiment may create the plan satisfying the preset condition for the plan and having the highest user preference, based on the various types of user information, e.g., the user profile 311, the plan history 312, and the score model 313.

For example, the condition for the plan may include various types of conditions to be considered to create the plan, e.g., a condition for foods to be excluded, a condition for a range of calories, and a condition for ranges of nutrients, based on the health status of the user.

For example, when the user requests a plan for weight loss through the interfaces 210 and 220 of FIG. 2, the plan creator 320 may create the plan satisfying the conditions for calories and nutrients and having the highest value of a score representing a user preference. The score according to an embodiment may be determined not only based on a user preference but also based on whether the one or more ingredients stored by the user may be consumed within the use-by dates according to the plan.

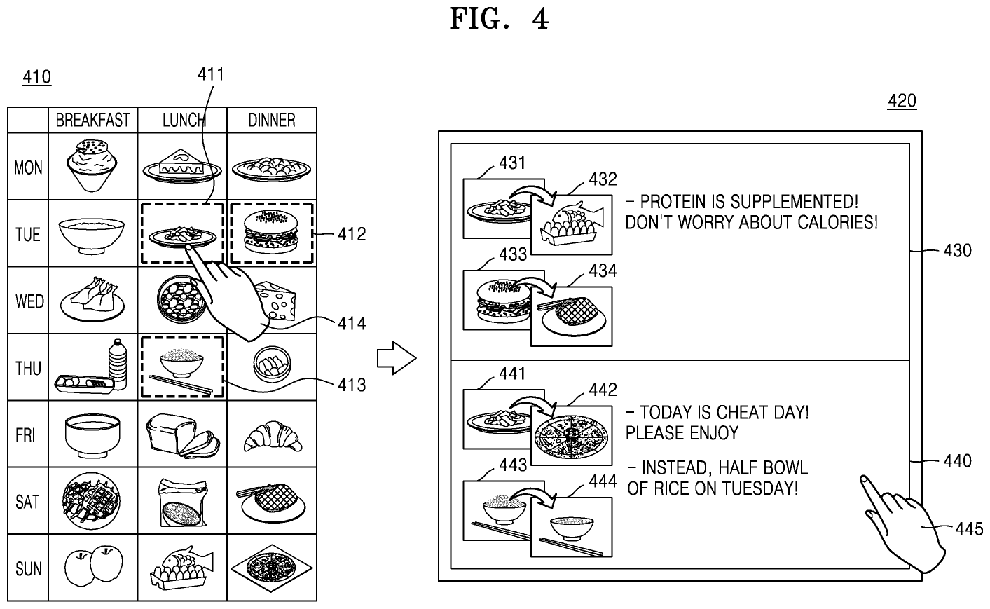

FIG. 4 is a diagram showing an example of replacing an item included in a plan, based on an input of a user, according to an embodiment of the disclosure.

Referring to FIG. 4, an electronic device 1000 may receive a user input 414 for requesting to replace an item 411 from among items included in a plan 410.

Through a user interface 420, the electronic device 1000 according to an embodiment may suggest one or more candidate plans 430 and 440 in which an item requested to be replaced is modified to other items based on the user input 414. According to an embodiment, according to a user selection 445, one of the suggested candidate plans 430 and 440 may be selected and the plan may be modified based on the selected candidate plan.

The candidate plans 430 and 440 according to an embodiment may include candidate items 432 and 442 which are significantly different from an item 431 or 441 requested to be replaced. For example, items, similarities of which to the item requested to be replaced are less than or equal to a reference value, may be suggested as candidate items.

The electronic device 1000 according to an embodiment may regard that replacement of an item is requested because the user does not prefer the item, and thus suggest, as candidate items, items having features significantly different from those of the item requested to be replaced.

According to an embodiment, the candidate items 432 and 442 may be determined as items having nutrients significantly different from those of the item 431 or 441 requested to be replaced. For example, when the item 431 or 441 requested to be replaced includes protein or calories less than or equal to a reference value, the candidate item 432 or 442 may be determined as the item 432 having more protein or the item 442 having more calories compared to the reference value.

According to an embodiment, the candidate plans 430 and 440 may additionally suggest to modify items 412 and 413 other than the item 411 requested to be replaced. Even when the item 411 requested to be replaced is replaced by items having significantly different features in the candidate plans 430 and 440, the electronic device 1000 according to an embodiment may additionally modify one or more items 412 and 413 to still satisfy a preset condition for the plan based on a goal or eating habits of the user according to the information input through the interfaces 210 and 220 of FIG. 2. For example, based on a goal of weight loss, when total calories of meals per day are preset to be within 1500 kcal, candidate plans in which one or more items of the plan are additionally modified to satisfy the preset condition may be suggested.

For example, in the first candidate plan 430, calories of the candidate item 432 may be higher than the calories of the item 431 requested to be replaced, and thus total calories of Tuesday meals may exceed a preset value. Therefore, not to exceed the preset calorie value, another item of the Tuesday meals, e.g., the item 412, may be additionally suggested to be changed to an item having less calories.

As another example, like the first candidate plan 430, in the second candidate plan 440, calories of the candidate item 442 may be higher than the calories of the item 441 requested to be replaced, and thus total calories of Tuesday meals may exceed a preset value. In the second candidate plan 440, to reduce calories of Thursday meals by the increase in total calories of the Tuesday meals, another item 413 or 443 of the Thursday meals may be additionally suggested to be changed to an item 444 having less calories.

According to an embodiment, the other item to be additionally modified in each candidate plan when the item 411 requested to be replaced is modified may be selected based on a preference of the user. For example, when the user prefers to reduce calories of dinner rather than breakfast, or less prefers an item placed as Tuesday dinner compared to an item placed as Tuesday breakfast, the candidate plan 430 may be suggested to change a dinner item 412 or 433 of the Tuesday meals to an item 434 having less calories. As another example, when user preferences for items placed as Tuesday breakfast and dinner are greater than or equal to a reference value, an item having a user preference less than the reference value from among items set for the other days may be selected and suggested as an item to be additionally changed.

According to an embodiment, without being limited to the above-described example, the item to be additionally changed may be selected to satisfy the preset condition with a higher user preference. The user preference according to an embodiment may be obtained as a score according to the score model 313, but is not limited thereto and may be obtained in various manners.

The candidate plans 430 and 440 according to an embodiment may be sequentially arranged based on final scores obtained for the candidate plans 430 and 440. According to an embodiment, the final scores of the candidate plans 430 and 440 may be obtained based on the items 432, 434, 442, and 444 suggested to be changed and included in the candidate plans 430 and 440.

For example, the final score of the candidate plan 430 may be obtained based on a plan including the items 432 and 434 suggested to be changed, instead of the items 431 and 433 requested to be replaced.

As another example, the candidate plans 430 and 440 may be sequentially arranged based on changes in score when the items 431, 433, 441, and 443 to be replaced are modified to the items 432, 434, 442, and 444 suggested to be changed.

For example, in the first candidate plan 430, when the item 431 is changed to the item 432, a score representing a degree of overlap between recipes from among scores of the plan may be increased by 3. When the item 433 is changed to the item 434, a score representing suitability of nutrients from among scores of the plan may be increased by 2. Therefore, a score of the first candidate plan 430 may be determined as being increased by a total of 5 compared to the score of the plan 410.

In the same manner, when a score of the second candidate plan 440 is determined as being increased by a total of 4 compared to the score of the plan 410, the first candidate plan 430, the score of which is increased more, may be provided to the user to be displayed with priority compared to the second candidate plan 440.

Without being limited to the above-described example, the candidate plans 430 and 440 may be provided to the user with priorities determined in various manners.

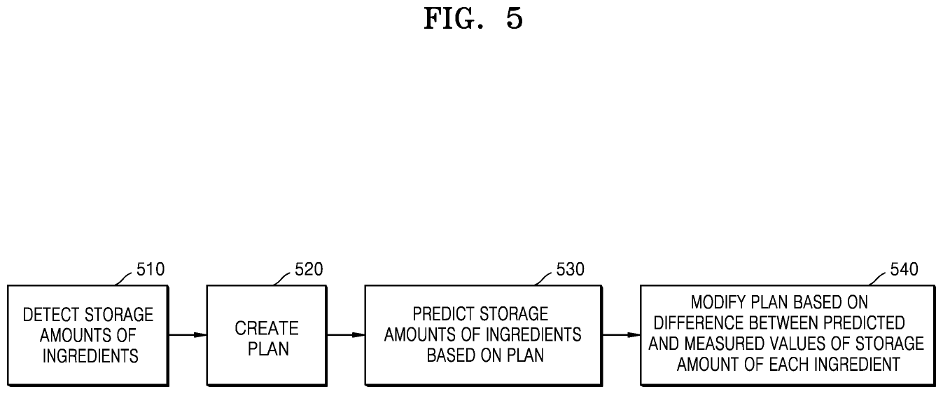

FIG. 5 is a block diagram showing an example of modifying a plan, according to an embodiment of the disclosure.

Referring to FIG. 5, when storage amounts of ingredients used for a plan are changed, an electronic device 1000 according to an embodiment may modify the plan already provided to a user, to consume all the ingredients by use-by dates of the ingredients. For example, when storage amounts of the ingredients predicted when the plan is created are significantly different from measured storage amounts of the ingredients, the electronic device 1000 may modify the plan suitably for the measured storage amounts of the ingredients.

In operation 510, the electronic device 1000 according to an embodiment may measure, in real time, storage amounts of one or more ingredients currently stored by the user. For example, the storage amounts of the ingredients of the user may be measured in real time by detecting weights, appearances, barcodes, or quick response (_{[A1]}QR) codes of the ingredients stored in a refrigerator. Without being limited to the above-described example, information about the storage amounts of the ingredients of the user may be obtained in various manners.

In operation 520, the electronic device 1000 according to an embodiment may create a plan based on the storage amounts of the ingredients measured in operation 510. The plan according to an embodiment may be created to consume all the ingredients by the use-by dates based on the storage amounts of the ingredients measured in operation 510. According to an embodiment, the plan may be created by determining consumption rates of the ingredients based on the use-by dates of the ingredients.

For example, the plan may be created in such a manner that the storage amounts of the ingredients are gradually reduced over time to zero before the use-by dates of the ingredients.

In operation 530, the electronic device 1000 according to an embodiment may predict how the storage amounts of the ingredients will be changed when the user performs activities based on the plan created in operation 520. For example, when the plan is a diet plan, storage amounts of foods may be predicted based on amounts of the foods used for recipes included in the plan.

After operation 530, the plan created in operation 520 may be provided to the user, and one or more ingredients may be consumed due to activities of the user. The electronic device 1000 according to an embodiment may measure, in real time, storage amounts of one or more ingredients consumed by the user based on the plan, and modify the plan as described below in relation to operation 540.

In operation 540, the electronic device 1000 according to an embodiment may modify the plan based on a difference between the storage amount of each ingredient predicted in operation 530, and the measured storage amount of the ingredient. When the difference between the predicted storage amount and the measured storage amount is greater than or equal to a reference value, the electronic device 1000 according to an embodiment may regard that the currently provided plan is not appropriate to consume the ingredient by the use-by date at an appropriate rate, and modify the plan.

According to an embodiment, the plan may be modified by re-determining the consumption rate of the ingredient to reduce the difference between the predicted storage amount and the measured storage amount at a subsequent point of time.

For example, when the currently measured storage amount of the ingredient is greater than the previously predicted storage amount, the ingredient may not be easily completely consumed within the use-by date. Therefore, the plan may be modified to increase an amount of the ingredient to be consumed based on the plan.

Otherwise, when the currently measured storage amount of the ingredient is less than the previously predicted storage amount, the ingredient may run out at a point of time much earlier than the use-by date. Therefore, the plan may be modified to reduce the amount of the ingredient to be consumed based on the plan.

According to an embodiment, the plan may or may not be modified based on selection of the user. According to an embodiment, when the user does not select to modify the plan, information for inducing appropriate action of the user may be provided to the user based on the storage amount of the ingredient. For example, when the measured storage amount of the ingredient is less than the predicted value, information indicating that additional purchase of the ingredient is required may be provided. Otherwise, when the measured storage amount of the ingredient is greater than the predicted value, information indicating that the ingredient may not be consumed by the use-by date may be provided to the user. Without being limited to the above-described example, information about the storage amount of the ingredient may be provided to the user in various forms.

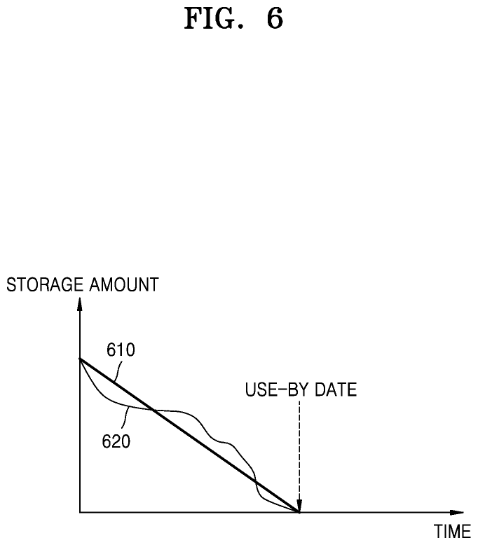

FIG. 6 is a graph showing an example of predicting a storage amount of an ingredient based on a plan, according to an embodiment of the disclosure.

Referring to FIG. 6, reference numeral 610 represents a change in a storage amount of an ingredient ideally consumed within a use-by date, and reference numeral 620 represents a change in a storage amount of the ingredient predicted to be consumed based on a plan. According to an embodiment, a plan may be created in such a manner that the value 620 approaches the value 610. For example, based on the amount of the ingredient predicted to be consumed based on the plan, the plan may be created in such a manner that the storage amount of the ingredient is changed closely to the value 610.

According to an embodiment, gradual consumption of the ingredient by a use-by date may be regarded as an ideal case, and the plan may be created accordingly. Without being limited to the above-described example, the plan for consuming the ingredient based on various types of changes in the storage amount of the ingredient may be created. For example, the plan may be created to gradually consume the ingredient by a point of time three days earlier than the use-by date.

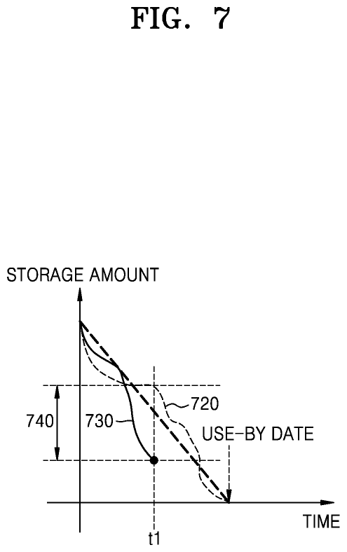

FIG. 7 is a graph showing an example of a measured value of a storage amount of an ingredient based on a plan, according to an embodiment of the disclosure.

Referring to FIG. 7, an electronic device 1000 according to an embodiment may measure a storage amount of an ingredient in real time as represented by reference numeral 730. The electronic device 1000 may compare the measured value 730 to a predicted value 720 of the storage amount of the ingredient and repeatedly determine whether a difference 740 therebetween is greater than or equal to a reference value.

According to an embodiment, at a point of time t1, the electronic device 1000 may detect that the difference 740 between the measured value 730 and the predicted value 720 is greater than or equal to the reference value, and thus modify a plan. According to an embodiment, when the measured value 730 is less than the predicted value 720 at the point of time t1, the electronic device 1000 modify the plan to reduce an amount of the ingredient to be consumed based on the plan, i.e., to reduce a rate at which the storage amount of the ingredient is reduced.

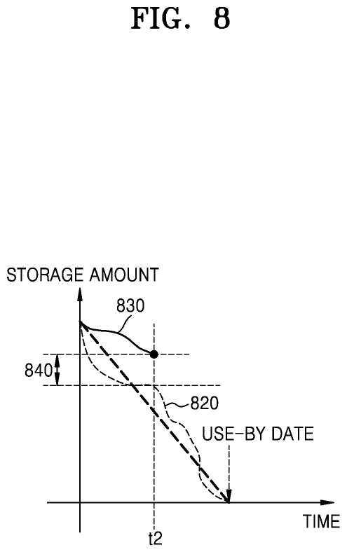

FIG. 8 is a graph showing an example of a measured value of a storage amount of an ingredient based on a plan, according to an embodiment of the disclosure.

Referring to FIG. 8, an electronic device 1000 according to an embodiment may measure a storage amount of an ingredient in real time as represented by reference numeral 830. The electronic device 1000 may compare the measured value 830 to a predicted value 820 of the storage amount of the ingredient and repeatedly determine whether a difference 840 therebetween is greater than or equal to a reference value.

According to an embodiment, at a point of time t2, the electronic device 1000 may detect that the difference 840 between the measured value 830 and the predicted value 820 is greater than or equal to the reference value, and thus modify a plan. According to an embodiment, when the measured value 830 is greater than the predicted value 820 at the point of time t2, the electronic device 1000 modify the plan to increase an amount of the ingredient to be consumed based on the plan, i.e., to increase a rate at which the storage amount of the ingredient is reduced.

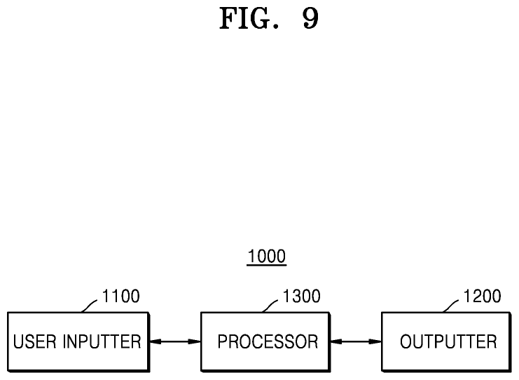

FIG. 9 is a block diagram of an electronic device according to an embodiment of the disclosure.

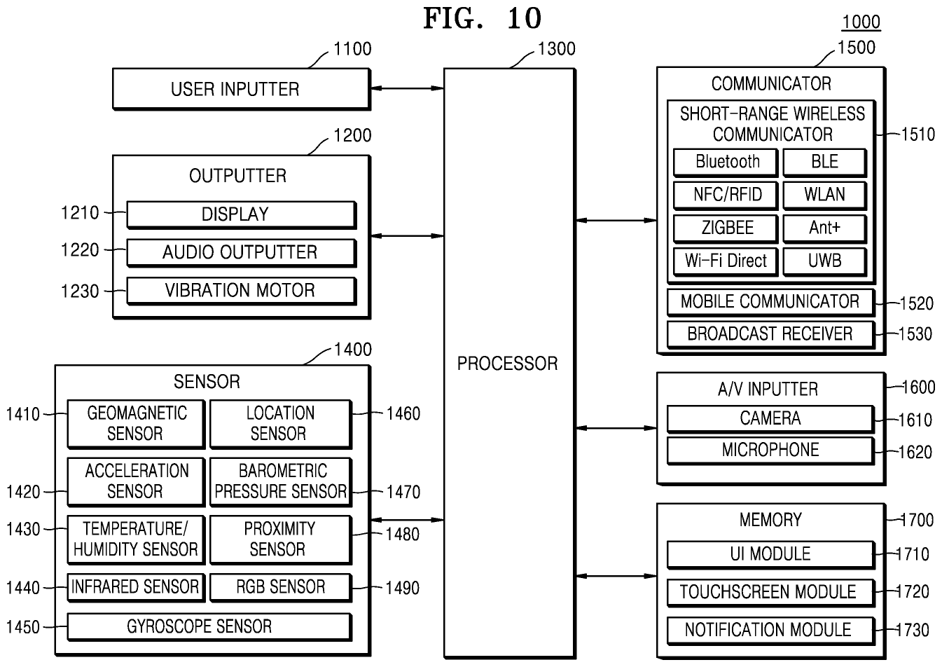

FIG. 10 is a detailed block diagram of an electronic device according to an embodiment of the disclosure.

Referring to FIG. 9, an electronic device 1000 may include an inputter 1100, a processor 1300, and an outputter 1200. However, not all elements illustrated in FIG. 9 are essential elements of the electronic device 1000. The electronic device 1000 may include a larger or smaller number of elements compared to those illustrated in FIG. 9.

Referring to FIG. 10, in addition to an inputter 1100, a processor 1300, and an outputter 1200, an electronic device 1000 according to an embodiment may further include a sensor 1400, a communicator 1500, an audio/video (A/V) inputter 1600, and a memory 1700.

The inputter 1100 may receive, from outside the electronic device 1000 (e.g., from a user), commands or data for controlling the electronic device 1000. For example, the inputter 1100 may include a keypad, a dome switch, a touchpad (e.g., a capacitive overlay, resistive overlay, infrared beam, surface acoustic wave, integral strain gauge, or piezoelectric touchpad), a jog wheel, or a jog switch, but is not limited thereto.

According to an embodiment, the inputter 1100 may receive an external input for creating a plan. The inputter 1100 may also receive an external input for modifying the plan. The electronic device 1000 according to an embodiment may modify an item included in the plan, based on the external input, and provide the modified plan to the user.

The outputter 1200 may output an audio signal, a video signal, or a vibration signal, and include a display 1210, an audio outputter 1220, and a vibration motor 1230. The outputter 1200 according to an embodiment may provide, to the user, the plan created or modified based on the input of the user.

The display 1210 displays data processed in the electronic device 1000. According to an embodiment, the display 1210 may display the plan created based on the input of the user.

Meanwhile, when a touchpad is layered on the display 1210 to configure a touchscreen, the display 1210 may be used not only as an output device but also as an input device. The display 1210 may include at least one of a liquid crystal display, a thin film transistor-liquid crystal display, an organic light-emitting diode, a flexible display, a three-dimensional (3D) display, and an electrophoretic display. Depending on implementation of the electronic device 1000, the electronic device 1000 may include two or more displays 1210.

The audio outputter 1220 outputs audio data received from the communicator 1500 or stored in the memory 1700. The vibration motor 1230 may output a vibration signal. When touch is input to the touchscreen, the vibration motor 1230 may also output a vibration signal. According to an embodiment, the audio outputter 1220 and the vibration motor 1230 may output information about the plan created based on the input of the user.

The processor 1300 generally controls overall operations of the electronic device 1000. For example, the processor 1300 may execute programs stored in the memory 1700 to generally control the user inputter 1100, the outputter 1200, the sensor 1400, the communicator 1500, and the A/V inputter 1600.

The electronic device 1000 may include at least one processor 1300. For example, the electronic device 1000 may include various types of processors, e.g., a central processing unit (CPU), a graphics processing unit (GPU), and a neural processing unit (NPU).

The processor 1300 may be configured to process commands of a computer program by performing basic arithmetic, logic, and input/output operations. The commands may be provided from the memory 1700 to the processor 1300, or be received through the communicator 1500 and be provided to the processor 1300. For example, the processor 1300 may be configured to execute commands based on program code stored in a recording device such as memory.

The processor 1300 according to an embodiment may create a plan including a plurality of items, based on a preset condition, and select one of the plurality of items based on an input of the user. The processor 1300 may obtain a first candidate item for replacing the selected item, and identify an item to be replaced to satisfy the preset condition from among the plurality of items, when the preset condition is not satisfied after the selected item is replaced by the first candidate item.

The first candidate item according to an embodiment may be obtained from among items, similarities of which to the item selected by the user are less than or equal to a reference value. According to an embodiment, the selected item is highly likely to be an item not preferred by the user, and thus may be replaced by an item having features different from those of the selected item by more than the reference value.

The processor 1300 may obtain a second candidate item for replacing the identified item, modify the plan based on the first and second candidate items, and provide the modified plan to the user.

According to an embodiment, the item identified to be replaced from among the plurality of items included in the plan may be identified from among items having user preferences less than or equal to a reference value from among the plurality of items included in the plan. According to an embodiment, because it is desirable to replace an item having a low user preference, the item to be replaced by the second candidate item may be determined from among the items having user preferences less than or equal to the reference value. The user preferences according to an embodiment may be determined based on the pre-trained score model 313, but is not limited thereto and may be determined in various manners.

When a plurality of first candidate items according to an embodiment are present, a plurality of candidate plans corresponding to the plurality of first candidate items may be provided to the user based on priorities. The priorities according to an embodiment may be determined based on changes in score when the items of the plan are replaced by the first and second candidate items. Alternatively, the priorities may be determined based on scores obtained for the modified plan when the items of the plan are replaced by the first and second candidate items.

The processor 1300 according to an embodiment may modify and provide the plan when a storage amount of an ingredient is changed. The processor 1300 according to an embodiment may obtain a plan including a plurality of items, predict, for one or more points of time, a storage amount of a first ingredient to be consumed based on the plan, and measure the storage amount of the first ingredient at the one or more points of time. The processor 1300 may detect that a difference between the measured and predicted values of the storage amount of the first ingredient at a first point of time from among the one or more points of time is greater than or equal to a reference value, and provide the plan by modifying the plan to reduce the difference between the measured and predicted values of the storage amount of the first ingredient after the first point of time.

The plan according to an embodiment may be created by determining a consumption rate of the first ingredient based on a use-by date of the first ingredient. However, when the difference between the measured and predicted values at the first point of time is greater than or equal to the reference value, the plan may be modified by re-determining the consumption rate of the first ingredient to reduce the difference after the first point of time. For example, the plan may be modified by re-determining the consumption rate of the first ingredient to minimize the difference between the predicted value and the measured value.

The sensor 1400 may detect a status of the electronic device 1000 or an ambient environment of the electronic device 1000, and transmit the detected information to the processor 1300.

The sensor 1400 may include at least one of a geomagnetic sensor 1410, an acceleration sensor 1420, a temperature/humidity sensor 1430, an infrared sensor 1440, a gyroscope sensor 1450, a location sensor (e.g., a global positioning system (GPS)) 1460, a barometric pressure sensor 1470, a proximity sensor 1480, and an RGB (or illuminance) sensor 1490, but is not limited thereto.

According to an embodiment, the information detected by the sensor 1400 may be obtained as user information. Without being limited thereto, the information detected by the sensor 1400 may be variously used to provide the plan.

The communicator 1500 may include one or more elements for enabling the electronic device 1000 to communicate with the server 2000 or an external device (not shown). For example, the communicator 1500 may include a short-range wireless communicator 1510, a mobile communicator 1520, and a broadcast receiver 1530.

The short-range wireless communicator 1510 may include a Bluetooth communicator, a Bluetooth low energy (BLE) communicator, a near-field communication (NFC) communicator, a wireless local area network (WLAN) (or wireless fidelity (Wi-Fi)) communicator, a Zigbee communicator, an infrared data association (IrDA) communicator, a Wi-Fi direct (WFD) communicator, a ultra-wideband (UWB) communicator, or an Ant+ communicator, but is not limited thereto.

The mobile communicator 1520 transmits and receives wireless signals to and from at least one of a base station, an external device, and a server in a mobile communication network. Herein, the wireless signals may include various types of data based on transmission and reception of voice call signals, video call signals, or text/multimedia messages.

The broadcast receiver 1530 receives broadcast signals and/or broadcast information through broadcast channels from outside. The broadcast channels may include satellite channels and terrestrial channels. Depending on implementation, the electronic device 1000 may not include the broadcast receiver 1530.

The communicator 1500 according to an embodiment may be used to receive, from the server 2000 or the external device, data required to create a plan.

The A/V inputter 1600 is used to input audio signals or video signals, and may include, for example, a camera 1610 and a microphone 1620. The camera 1610 may obtain still images or image frames of a video by using an image sensor in a video call mode or a camera mode. The images captured by the image sensor may be processed through the processor 1300 or a separate image processor (not shown).

The microphone 1620 receives an external audio signal and processes the same into electrical voice data. For example, the microphone 1620 may receive a voice input of the user for requesting to create a plan.

The memory 1700 may store programs for processing and control operations of the processor 1300, or store data input to or to be output from the electronic device 1000.

The memory 1700 according to an embodiment may store data required to create a plan. For example, the memory 1700 may store various types of user information, e.g., user profile information, health status information, ambient environment information, and plan history information. The memory 1700 may also store a score model for obtaining a score of the plan. The score according to an embodiment may represent a preference of the user for the plan. The electronic device 1000 according to an embodiment may provide a plan having a high user preference to the user based on the score of the plan.

The memory 1700 according to an embodiment may store information about ingredients used for the plan, for example, predicted values and measured values of storage amounts of the ingredients.

The memory 1700 may include at least one type of storage medium from among flash memory, a hard disk, a multimedia card micro, a memory card (e.g., a secure digital (SD) or extreme digital (XD) memory card), random access memory (RAM), static RAM (SRAM), read-only memory (ROM), electrically erasable programmable ROM (EEPROM), programmable ROM (PROM), magnetic memory, a magnetic disc, and an optical disc.

The programs stored in the memory 1700 may be classified into a plurality of modules, e.g., a user interface (UI) module 1710, a touchscreen module 1720, and a notification module 1730, depending on functions thereof.

The UI module 1710 may provide a specialized UI or graphical user interface (GUI) connected to the electronic device 1000, per application. The touchscreen module 1720 may detect a touch gesture of the user on a touchscreen, and transmit information about the touch gesture to the processor 1300. The touchscreen module 1720 according to an embodiment may recognize and analyze touch code. The touchscreen module 1720 may be configured as separate hardware including a controller.

Various sensors may be provided in or near the touchscreen to detect touch or proximity touch on the touchscreen. The sensors for detecting touch on the touchscreen may include, for example, a tactile sensor. The tactile sensor refers to a sensor for detecting contact of a certain object like or better than a human. The tactile sensor may detect various types of information, e.g., roughness of a contact surface, hardness of a contact object, and temperature at a contact point.

The touch gesture of the user may include tap, touch & hold, double tap, drag, pan, flick, drag & drop, swipe, etc.

The notification module 1730 may generate a notification signal to notify that an event of the electronic device 1000 has occurred.

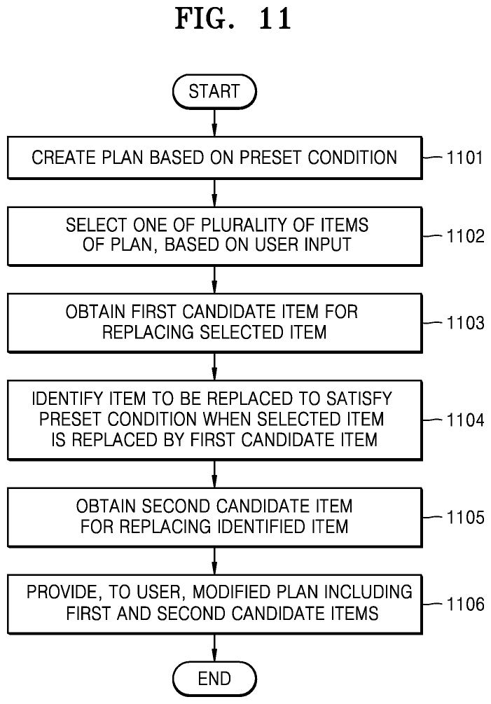

FIG. 11 is a flowchart of a method of providing a plan, according to an embodiment of the disclosure.

Referring to FIG. 11, in operation 1101, an electronic device 1000 according to an embodiment may create a plan including a plurality of items, based on a preset condition.

The electronic device 1000 according to an embodiment may set a condition for creating a plan, based on an external input (e.g., an input by a user), and create a plan satisfying the set condition and determined as being most suitable for the user. For example, the electronic device 1000 may set the condition of the plan, based on the information input through the interfaces 210 and 220 of FIG. 2, and create the plan by obtaining a final score representing a user preference for the plan, based on a pre-trained score model.

In operation 1102, the electronic device 1000 according to an embodiment may select one of the plurality of items included in the plan created in operation 1101, based on an external input. According to an embodiment, the user may select one item in operation 1102 to replace an item not preferred by the user from among the items included in the plan created in operation 1101.

In operation 1103, the electronic device 1000 according to an embodiment may obtain a first candidate item for replacing the item selected in operation 1102. The first candidate item according to an embodiment may be obtained as an item, similarity of which to the selected item is less than or equal to a reference value. Because the item selected by the user in operation 1102 is highly likely to be an item having features not preferred by the user, an item having features significantly different from those of the selected item may be obtained as the first candidate item.

The similarity between the items according to an embodiment may be determined using a model pre-trained to extract features of items and determine similarity between the items, but is not limited thereto and may be determined in various manners.

In operation 1104, the electronic device 1000 according to an embodiment may identify an item to be additionally replaced in the plan to still satisfy the preset condition for the plan when the item selected in operation 1102 is replaced by the first candidate item.

According to an embodiment, when the item selected in operation 1102 is replaced based on an input of the user, the plan may not satisfy the preset condition. The electronic device 1000 according to an embodiment may additionally replace another item in the plan to still satisfy the preset condition even when the item selected in operation 1102 is replaced.

According to an embodiment, the item identified in operation 1104 may be identified based on whether a user preference thereof is less than or equal to a reference value. The user preference according to an embodiment may be determined based on a final score obtained by the above-described score model 313. For example, the item to be replaced may be identified based on whether the final score representing a user preference for the identified item is less than or equal to the reference value.

According to an embodiment, by replacing the item having a low user preference, the plan more suitable for the user may be created. Therefore, according to an embodiment, the item having a low user preference from among the plurality of items may be identified as the item to be additionally replaced.

In operation 1105, the electronic device 1000 according to an embodiment may obtain a second candidate item for replacing the identified item. According to an embodiment, the second candidate item capable of satisfying the preset condition for the plan may be obtained.

When a plurality of first candidate items according to an embodiment are present, a plurality of candidate plans corresponding to the plurality of first candidate items may be provided to the user based on priorities. The priorities according to an embodiment may be determined based on changes in score when the items included in the plan are replaced according to the plurality of candidate plans, or scores of the plan modified according to the plurality of candidate plans.

For example, a candidate plan corresponding to a larger change in score of the plan from among the plurality of candidate plans may have a higher priority. As another example, a candidate plan corresponding to a higher final score of the modified plan from among the plurality of candidate plans may have a higher priority.

The plurality of candidate plans according to an embodiment are provided to the user with priorities based on final scores, and thus a candidate plan determined as having a higher user preference may be preferentially provided to the user.

In operation 1106, the electronic device 1000 according to an embodiment may provide, to the user, a modified plan including the first and second candidate items. For example, the modified plan in which the item selected in operation 1102 and the item identified in operation 1104 are respectively replaced by the first and second candidate items may be provided to the user.

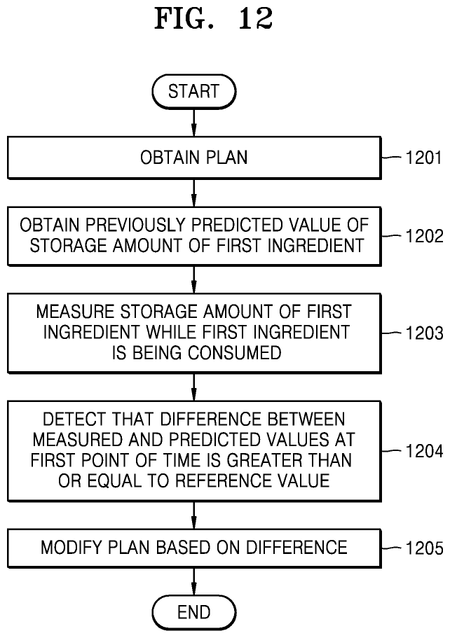

FIG. 12 is a flowchart of a method of providing a plan, according to an embodiment of the disclosure.

Referring to FIG. 12, in operation 1201, an electronic device 1000 according to an embodiment may obtain a plan including a plurality of items. The plan according to an embodiment may be, for example, a diet plan including recipes. A user according to an embodiment may consume one or more ingredients by making meals based on the plan.

According to an embodiment, the plan may be created considering not only a user preference but also storage amounts of ingredients used for the plan. According to an embodiment, the plan may be created by determining a consumption rate of a first ingredient based on a use-by date of the first ingredient. According to an embodiment, the plan may be modified and provided to consume each ingredient within an appropriate use-by date based on rates at which one or more ingredients are consumed by the user.

In operation 1202, the electronic device 1000 according to an embodiment may obtain, for one or more points of time, a previously predicted value of a storage amount of the first ingredient from among the one or more ingredient. According to an embodiment, the previously predicted value of the storage amount of the first ingredient may refer to a predicted storage amount of the first ingredient when the user consumes the first ingredient based on the plan obtained in operation 1201.

In operation 1203, the electronic device 1000 according to an embodiment may measure the storage amount of the first ingredient at the one or more points of time while the first ingredient is being consumed based on the plan. According to an embodiment, the value measured in operation 1203 may be compared to the value predicted in operation 1202.

The electronic device 1000 according to an embodiment may compare the value measured in operation 1203 to the value predicted in operation 1202 at the first point of time from among the one or more points of time, and determine whether a difference between the measured and predicted values is greater than or equal to a reference value, in operation 1204.

In operation 1205, the electronic device 1000 according to an embodiment may modify the plan based on the difference detected in operation 1204. According to an embodiment, the plan may be modified to reduce the difference between the measured and predicted values of the storage amount of the first ingredient after the first point of time.

For example, the plan may be modified by re-determining the consumption rate of the first ingredient to minimize the difference after the first point of time. For example, when the measured value is greater than the predicted value, the plan may be modified to consume the first ingredient faster. Otherwise, when the measured value is less than the predicted value, the plan may be modified to consume the first ingredient more slowly.

According to an embodiment, even when a user changes a plan, a plan capable of allowing the user to achieve a preset goal or stay healthy may be provided.

According to an embodiment, the plan may be appropriately modified and provided based on changes in storage amounts of ingredients.

The machine-readable storage medium may be provided in the form of a non-transitory storage medium. When the storage medium is ‘non-transitory’, it means that the storage medium is tangible and does not include signals (e.g., electromagnetic waves), and it does not limit that data is semi-permanently or temporarily stored in the storage medium. For example, the ‘non-transitory storage medium’ may include a buffer for temporarily storing data.

According to an embodiment, the method according to various embodiments of the disclosure may be included and provided in a computer program product. The computer program product may be traded as a product between a seller and a buyer. The computer program product may be distributed in the form of a machine-readable storage medium (e.g., a compact disc read only memory (CD-ROM)), or be electronically distributed (e.g., downloaded or uploaded) via an application store (e.g., Play Store™) or directly between two user devices (e.g., smartphones). For electronic distribution, at least a part of the computer program product (e.g., a downloadable app) may be temporarily generated or be at least temporarily stored in a machine-readable storage medium, e.g., a memory of a server of a manufacturer, a server of an application store, or a relay server.

As used herein, the term “unit” may indicate a hardware component such as a processor or a circuit, and/or a software component executed by the hardware component such as the processor.

The above descriptions of the disclosure are provided for the purpose of illustration, and it will be understood by one of ordinary skill in the art that various changes in form and details may be made therein without departing from the scope of the disclosure. Therefore, it should be understood that the afore-described embodiments should be considered in a descriptive sense only and not for purposes of limitation. For example, each component described to be of a single type can be implemented in a distributed manner and, likewise, components described as being distributed can be implemented in a combined manner.

While the disclosure has been shown and described with reference to various embodiments thereof, it will be understood by those skilled in the art that various changes in form and details may be made therein without departing from the spirit and scope of the disclosure as defined by the appended claims and their equivalents.

1. A method, performed by an electronic device, of providing a plan, the method comprising:
obtaining a plan including a plurality of items;
predicting, for one or more points of time, a storage amount of a first ingredient to be consumed over time based on the plan;
measuring the storage amount of the first ingredient at the one or more points of time;
detecting a difference between the measured and predicted values at a first point of time from among the one or more points of time; and
revising the plan by modifying the plan to reduce the difference after the first point of time, in response to the detected difference being greater than or equal to a reference value.

2. The method of claim 1, wherein the plan is created by determining a consumption rate of the first ingredient based on a use-by date of the first ingredient.

3. The method of claim 1, wherein the plan is modified by re-determining a consumption rate of the first ingredient to reduce the difference after the first point of time.

4. The method of claim 1,
wherein a use-by date of the first ingredient is changeable in real time depending on a storage state of the first ingredient, and
wherein the plan is modified based on the changed use-by date of the first ingredient.

5. A method, performed by an electronic device, of providing a plan, the method comprising:
creating a plan including a plurality of items, based on a preset condition;
selecting one of the plurality of items based on an external input signal;
obtaining a first candidate item for replacing the selected item;
identifying an item to be replaced to satisfy the preset condition from among the plurality of items, in response to the preset condition not being satisfied after the selected item is replaced by the first candidate item;
obtaining a second candidate item for replacing the identified item; and
modifying the plan based on the first and second candidate items, and displaying the modified plan.

6. The method of claim 5, wherein an item having a user preference less than or equal to a reference value from among the plurality of items is identified as the item to be replaced to satisfy the preset condition.

7. The method of claim 5,
wherein, in response to a plurality of first candidate items being present, information about a plurality of candidate plans respectively including the plurality of first candidate items and second candidate items corresponding to the plurality of first candidate items is displayed based on priorities of the candidate plans, and
wherein the priorities are determined based on scores representing user preferences for the plurality of candidate plans.

8. An electronic device for providing a plan, the electronic device comprising:
an inputter for receiving an external input signal;
a memory for storing one or more instructions; and
at least one processor for executing the one or more instructions stored in the memory,
wherein the at least one processor, when executing the one or more instructions, is configured to:
obtain a plan including a plurality of items,
predict, for one or more points of time, a storage amount of a first ingredient to be consumed over time based on the plan,
measure the storage amount of the first ingredient at the one or more points of time,
detects a difference between the measured and predicted values at a first point of time from among the one or more points of time, and
revise the plan by modifying the plan to reduce the difference after the first point of time, in response to the detected difference being greater than or equal to a reference value.

9. The electronic device of claim 8, wherein the plan is created by determining a consumption rate of the first ingredient based on a use-by date of the first ingredient.

10. The electronic device of claim 8, wherein the plan is modified by re-determining a consumption rate of the first ingredient to reduce the difference after the first point of time.

11. The electronic device of claim 8,
wherein a use-by date of the first ingredient is changeable in real time depending on a storage state of the first ingredient, and
wherein the plan is modified based on the changed use-by date of the first ingredient.

12. An electronic device for providing a plan, the electronic device comprising:
an inputter for receiving an external input signal;
a memory for storing one or more instructions; and
at least one processor for executing the one or more instructions stored in the memory,
wherein the at least one processor, when executing the one or more instructions, is configured to:
create a plan including a plurality of items, based on a preset condition,
select one of the plurality of items based on the external input signal,
obtain a first candidate item for replacing the selected item,
identify an item to be replaced to satisfy the preset condition from among the plurality of items, in response to the preset condition not being satisfied after the selected item is replaced by the first candidate item,
obtain a second candidate item for replacing the identified item, and
modify the plan based on the first and second candidate items, and provides the modified plan.

13. The electronic device of claim 12, wherein an item having a user preference less than or equal to a reference value from among the plurality of items is identified as the item to be replaced to satisfy the preset condition.

14. The electronic device of claim 12,
wherein, in response to a plurality of first candidate items being present, information about a plurality of candidate plans respectively including the plurality of first candidate items and second candidate items corresponding to the plurality of first candidate items is displayed based on priorities of the candidate plans, and
wherein the priorities are determined based on scores representing user preferences for the plurality of candidate plans.

15. A computer-readable recording medium having recorded thereon a computer program for executing the method of claim 1.

16. The electronic device of claim 12, wherein the preset condition comprises a calorie range.

17. The electronic device of claim 12, wherein the preset condition is based on a user goal.

18. The electronic device of claim 17, wherein the user goal comprises one of weight loss, muscle gain, detox, child nutrition, infant nutrition, pregnancy nutrition, or balanced nutrition.
